(19) United States
(12) Patent Application Publication
(10) Pub. No.: US 2024/0207458 A1
(43) Pub. Date: Jun. 27, 2024

(54) TARGETED ALPHA PARTICLE THERAPY FOR SOMATOSTATIN RECEPTOR 2 POSITIVE NEUROENDOCRINE TUMORS AND METASTASES

(71) Applicant: H. LEE MOFFITT CANCER CENTER AND RESEARCH INSTITUTE, INC., Tampa, FL (US)

(72) Inventors: David MORSE, Valrico, FL (US); Haitao JI, Tampa, FL (US); Thaddeus WADAS, Iowa City, IA (US); Darpan PANDYA, Coralville, IA (US)

(21) Appl. No.: 18/556,938

(22) PCT Filed: Apr. 26, 2022
(86) PCT No.: PCT/US2022/026318

§ 371 Date: Oct. 24, 2023

Related U.S. Application Data
(60) Provisional application No. 63/179,798, filed on Apr. 26, 2021.

(51) Int. Cl. A61K 51/08; A61P 35/00

(52) U.S. Cl. CPC A61K 51/083; A61P 35/00

(57) ABSTRACT

Disclosed are TATE derivatives having a linker between the TATE moiety and a macrocyclic radionuclide chelating moiety. Methods of synthesis and use are also disclosed. The compounds preferably exhibit a ratio of kidney: liver uptake by a subject within 24 hours of 5 or less, 3 or less, 1 or less, 0.5 or less, preferably from 2:1 to 1:2.

### CROSS-REFERENCE TO RELATED APPLICATIONS

This application claims the benefit of priority to U.S. Provisional Application No. 63/179,798, filed Apr. 26, 2021, the disclosure of which is incorporated herein by reference in its entirety.

### STATEMENT OF GOVERNMENT FUNDING

This invention was made with Government support under Grant No. CA076292 awarded by the National Institutes of Health. The Government has certain rights in the invention.

### BACKGROUND

The efficacy and safety of 177Lu-DOTATATE in patients with advanced, progressive, somatostatin-receptor positive midgut neuroendocrine tumors have been reported. The treatment resulted in longer progression-free survival and a higher response rate relative to high-dose octreotide LAR (J. Strosberg, et al., N Engl J Med 376, 125-135 (2017)). An increased time to quality of life deterioration was also reported (J. Strosberg, et al., J Clin Oncol, JCO2018785865 (2018)). While these results are encouraging, 177Lu is a β-particle emitting radionuclide and there is recent evidence that systemically administered targeted α-particle emitting radiotherapies (TATs) have greater efficacy and reduced toxicity relative to targeted β-particle emitting therapies (TBTs) (M. Miederer, et al., Clin Cancer Res 14, 3555-3561 (2008); H. Song, et al., Cancer Res 69, 8941-8948 (2009); H. Andersson, et al., Anticancer Res 21, 409-412 (2001); M. Essler, et al., Eur J Nucl Med Mol Imaging 39, 602-612 (2012); D. Milenic, et al., Cancer Biother Radiopharm 19, 135-147 (2004); D. Wild, et al., Cancer Res 71, 1009-1018 (2011)). This is particularly true for ²²⁵Ac-based TATs due to the long 10 day half-life which allows for internalization of the therapy into tumor cells and systemic clearance prior to when the majority of α-emissions take place and due to the four-alpha particles emitted in the decay chain, where the daughter emissions occur rapidly following the initial 225 Ac α-decay (H. Song, et al., Cancer Res 69, 8941-8948 (2009); M. Essler, et al., Eur J Nucl Med Mol Imaging 39, 602-612 (2012); C. Kratochwil, et al., J Nucl Med 59, 795-802 (2018)). Hence, 225 Ac has been described as an in vivo α-particle generator (M. Miederer, et al., Clin Cancer Res 14, 3555-3561 (2008)). Compared to β-particles, α-particles have a 430× greater linear energy transfer and a shorter range in solid tissue, <100 μm for α-emissions relative to a few mm for β-emissions. Unlike β-emissions, α-emissions do not rely on generation of free-radicals to generate DNA damage. Instead, the energy deposited is sufficient to directly cause DNA double-strand breaks (J. Nonnekens, et al., Cancer Biother Radiopharm 32, 67-73 (2017)). This enables TATs to evade a common mechanism of radiation resistance (free radical scavenging).

In demonstration of these advantages, ²²⁵Ac-DOTATOC was compared to 177Lu-DOTATOC in the treatment of rat pancreatic exocrine tumor xenografts in immunocompromised mice and the TAT demonstrated greater efficacy and lower toxicity relative to the TBT (M. Miederer, et al., Clin Cancer Res 14, 3555-3561 (2008)).

DOTATATE is a compound made by conjugation of the metal chelator DOTA to the somatostatin-receptor (SSTR2) specific octreotate peptide and DOTATOC is the SSTR2-specific octreotide conjugate. ¹⁷⁷Lu-DOTATATE has been compared with ¹⁷⁷Lu-DOTATOC in patients and a greater tumor residence time was observed for ¹⁷⁷Lu-DOTATATE, indicating that DOTATATE may be a superior targeting ligand for delivery of radiotherapy (J. P. Esser, et al., Eur J Nucl Med Mol Imaging 33, 1346-1351 (2006)).

When compared to ¹⁷⁷Lu-DOTATATE, the targeted alpha particle therapy (TAT) ²²⁵Ac-DOTATATE can demonstrate significantly increased efficacy and decreased toxicity in the treatment of somatostatin receptor 2 (SSTR2) positive neuroendocrine tumors. However, TATs are known to involve elevated renal toxicity relative to beta emitting derivatives. What are thus needed are new DOTATATE derivatives that have low toxicity and high efficacy relative to DOTATATE. The compositions and methods disclosed herein address these and other needs.

### SUMMARY

In accordance with the purposes of the disclosed materials and methods, as embodied and broadly described herein, the disclosed subject matter, in one aspect, relates to compounds, compositions and methods of making and using compounds and compositions. In specific aspects, the disclosed subject matter relates to cancer therapy and to anti-cancer compounds. More specifically, the subject matter disclosed herein relates to TATE derivatives having a linker between the TATE moiety and a macrocyclic radionuclide chelating moiety. In further examples, disclosed are DOTA-linker-TATE derivatives. Methods of synthesis and use are also disclosed.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects described below.

FIG. 1 shows a library of DOTA-linker-TATE derivatives of DOTATATE.

FIG. 2A shows the HPLC spectrum of peptide 1. FIG. 2B shows the mass spectrometry spectrum of peptide 1.

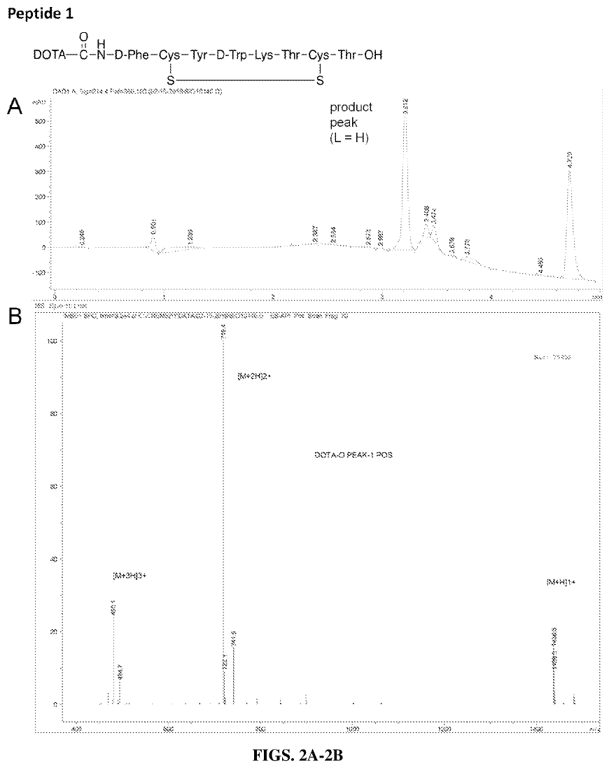

FIG. 3 shows competition binding assay results for 5 compounds using Eu³⁺-DOTATATE as the competing ligand.

FIG. 4 shows biodistribution of 225Ac-DOTA-liniker-TATE derivatives in BALB/c mice. Note the different Y-axis scales.

FIG. 5 shows graphs with the binding affinity of compound 14 (ZW-7-123; ¹³⁹La-DOTA-D-Phe-D-Ala-TATE) for the SSTR2 receptor and EC₅₀ values.

FIG. 6 shows graphs with the binding affinity of compound 15 (ZW-7-124; ¹³⁹La-DOTA-D-Phe-D-Leu-TATE) for the SSTR2 receptor and EC₅₀ values.

### DETAILED DESCRIPTION

The materials, compounds, compositions, and methods described herein may be understood more readily by reference to the following detailed description of specific aspects of the disclosed subject matter and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an inhibitor” includes mixtures of two or more such inhibitors, and the like.

“Optional” or “optionally” means that the subsequently described event or circumstance can or cannot occur, and that the description includes instances where the event or circumstance occurs and instances where it does not.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

By “reduce” or other forms of the word, such as “reducing” or “reduction,” is meant lowering of an event or characteristic (e.g., tumor growth, metastasis). It is understood that this is typically in relation to some standard or expected value, in other words it is relative, but that it is not always necessary for the standard or relative value to be referred to. For example, “reduces tumor growth” means decreasing the amount of tumor cells relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of: alleviation of one or more symptoms (such as tumor growth or metastasis), diminishment of extent of cancer, stabilized (i.e., not worsening) state of cancer, delaying spread (e.g., metastasis) of the cancer, delaying occurrence or recurrence of cancer, delay or slowing of cancer progression, amelioration of the cancer state, and remission (whether partial or total).

The term “patient” preferably refers to a human in need of treatment with an anti-cancer agent or treatment for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with an anti-cancer agent or treatment.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

### Compositions

Radiolabeled DOTATATE, e.g., ⁶⁸Ga-DOTATATE has a reported log P value of −3.69, which is in the range of high renal toxicity for TAT. Disclosed herein are modifications to the chemical properties of the linker that connects a radionuclide chelator (such as DOTA) to the peptidic targeting moiety, which modifications to the linker resulted in altered lipophilicity (Log D) while retaining high binding affinity and proportionally altering the clearance route, i.e. renal vs. hepatic. Specifically, disclosed herein are peptidic TAT that provides a balance between renal and liver clearance, having low toxicities and high efficacy. More specifically, disclosed herein are DOTA-linker-TATE derivatives that can have low toxicity and high efficacy relative to DOTATATE.

In some aspects, disclosed are compounds represented by Formula I:

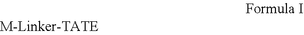

wherein

M is a macrocyclic radionuclide chelating moiety with the radionuclide optionally chelated therein;

Linker is comprises a non-polar bridging moiety; and TATE is

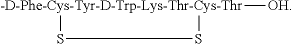

In some examples of Formula I, the linker is not phenylalanine alone.

The distribution coefficient, log D at pH 7.4, of the compounds is typically greater than −3.0. Preferably, the compound has a log D7.4 of −2.8 or greater, −2.7 or greater, −2.6 or greater, −2.5 or greater, −2.4 or greater, −2.3 or greater, −2.2 or greater, −2.1 or greater, or −2.0 or greater. In some embodiments, the compound has a log D7.4 of from −2.8 to less than 4, from −2.7 to less than 4, from −2.6 to less than 4, from −2.8 to less than 2, from −2.7 to less than 2, or from −2.6 to less than 2. The log D7.4 values can be calculated or experimentally determined using methods well known in the art.

The partition coefficient, log P, of the compounds is typically greater than −3.0. Preferably, the compound has a log P of −2.8 or greater, −2.7 or greater, −2.6 or greater, −2.5 or greater, −2.4 or greater, −2.3 or greater, −2.2 or greater, −2.1 or greater, or −2.0 or greater. In some embodiments, the compound has a log P of from −2.8 to less than 4, from −2.7 to less than 4, from −2.6 to less than 4, from −2.8 to less than 2, from −2.7 to less than 2, or from −2.6 to less than 2. The log P values can be calculated or experimentally determined using methods well known in the art.

### Macrocyclic Radionuclide Chelating Moiety

In specific examples, the macrocyclic radionuclide chelating moiety, M, can be 2,2′,2″-(10-(2-((2,5-dioxopyrrolidin-1-yl)oxy)-2-oxoethyl)-1,4,7,10-tetraazacyclododecane-1,4,7-triyl)triacetic acid (DOTA)-based chelators, such as such as DTPA (diethylene triamine pentaacetic acid), DOTP (1,4,7,10-Tetraazacyclododecane-1,4,7,10-tetra(methylene phosphonic) acid), DOTMA, (1R, 4R, 7R, 10R)-α′α″α″-Tetramethyl-1,4,7,10-tetraazacyclododecane-1,4,7,10-tetraacetic acid) tetrasodium salt, TETA, (1,4,8,11-Tetraazacyclotetradecane-1,4,8,11-tetraacetic acid), DOTAM (1,4,7,10-Tetrakis(carbamoylmethyl)-1,4,7,10-tetraazacyclododecane), CB-TE2A (1,4,8,11-tetraazabicyclo[6.6.2]hexadecane-4,11-dicetic acid), and NOTA ((1,4,7-triazacyclononane-N,N′,N″-triacetic acid), and derivatives or a combination thereof. Examples of other macrocyclic radionuclide chelating moieties can include DOTAGA, TRITA, DO3A-Nprop, tetra-tBu-DTPA, p-SCN-Bz-DTPA, MX-DTPA, CHX-DTPA, TACN, TACN-TM, H3NOKA, NODASA, NODAGA, NOTP, NOTPME, PrP9, TRAP, NOPO, TETA, HBED, DFO, EDTA, 6SS, B6SS, PLED, TAME, YM103, NTP(PRHP)3, H2dedpa, H2dp-bb-NCS, H2dp-N-NCS, (4,6-MeO2sal)2-BAPEN and citrate. In a specific example, M is DOTA (2,2′,2″-(10-(2-((2,5-dioxopyrrolidin-1-yl)oxy)-2-oxoethyl)-1,4,7,10-tetraazacyclododecane-1,4,7-triyl)triacetic acid).

As described herein, the macrocyclic radionuclide chelating moiety, M, can include a chelated radioactive metal. The “radioactive metal” encompasses all radioactive metal ions suitable for use in medical imaging or radionuclides therapy. The radioactive metals are typically radioisotopes or radionuclides such as: copper-64, gallium-68, gallium-67, gallium-66, lutecium-177, yttrium-86, yttrium-90, indium-114, indium-111, scandium-47, scandium-44, scandium-43, zirconium-89, bismuth-213, bismuth-212, actinium-225, lead-212, rhenium-188, rhenium-186, rubidium-82 and the like. The “radioactive metal as used herein also encompasses metal linked to a radioactive species such as for example fluorine-18-based metallic fluorides.

In some examples, the macrocyclic radionuclide chelating moiety includes a chelated radionuclide selected from ⁸⁹Zr, ⁴⁴Sc, ¹¹¹In, ⁹⁰Y, ⁶⁸Ga, ¹⁷⁷Lu, ⁹⁹mTc, ⁶⁴Cu, ⁶⁷Cu, ¹⁵³Gd, ¹⁵⁵Gd, ¹⁵⁷Gd, ²¹³Bi, or ²²⁵Ac. In preferred examples, the radionuclide can be ²²⁵Ac.

### Linker

As described herein, modifications to the chemical properties of the linker that connects the macrocyclic radionuclide chelating moiety to the targeting moiety (TATE) can provide altered lipophilicity (Log D) while retaining high binding affinity as well as proportional alteration of the clearance route of the compounds, i.e. renal vs. hepatic. The specific linker used in the compounds can also serve to maintain the proper binding conformation of the targeting moiety in the receptor as well as adjust the physicochemical property of the resulting linker.

In some examples, the compound can have a structure represented by Formula I-A:

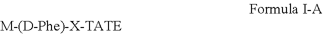

wherein
M is the macrocyclic radionuclide chelating moiety;
D-Phe is D-phenylalanine and maintains proper binding conformation of the compound to a receptor; and
X is from 1 to 100 atoms in length and comprises an organic moiety to adjust a physicochemical property of the compound.

In specific examples, the compound can be represented by the formula

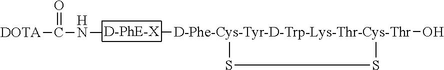

wherein D-Phe is preserved to maintain the proper binding conformation; and X is any suitable organic moiety to adjust the physicochemical property of the resulting compound.

The linker or X as described herein can be any suitable organic moiety and preferably is from 1 to 100 atoms in length and comprises a non-polar bridging moiety. In some example, the linker or X can be selected from a natural or non-natural amino acid, a peptide or peptide mimetic, a polyethylene oxide, an optionally substituted amide, an optionally substituted ester, an optionally substituted C₁-C₁₂ alkyl, an organic moiety as further described below, or a combination; wherein the optional substituent can include one or more substituents selected from halogen, alkoxyl, alkyl, alkenyl, cycloalkyl, cycloalkenyl, heterocycloalkyl, aryl, heteroaryl, amine, cyano, nitro, hydroxyl, carbonyl (C═O), acyl, carboxylic acid (—COOH), or amide (—CONH₂). In some examples, X can be an amino acid unit with either D or L stereochemistry.

The distribution coefficients, log D, of the linker or X is typically greater than −0.3. Preferably, the linker or X has a log D of −0.2 or greater, −0.1 or greater, 0 or greater, 0.1 or greater, 0.2 or greater, 0.3 or greater, 0.4 or greater, 0.5 or greater, 0.6 or greater, 0.7 or greater, 0.8 or greater, 0.9 or greater, 1 or greater, 1.1 or greater, 1.2 or greater, 1.2 or greater, 1.4 or greater, 1.5 or greater, 1.6 or greater, 1.7 or greater, 1.8 or greater, 1.9 or greater, or 2.0 or greater. In some embodiments, the linker or X has a log D of from −0.3 to 4, from −0.2 to 4, from −0.1 to 4, from 0.1 to 4, from 0.1 to 3, or from 0.1 to 2.

The partition coefficient, log P, of the linker or X is typically greater than −0.3. Preferably, the linker or X has a log P of −0.2 or greater, −0.1 or greater, 0 or greater, 0.1 or greater, 0.2 or greater, 0.3 or greater, 0.4 or greater, 0.5 or greater, 0.6 or greater, 0.7 or greater, 0.8 or greater, 0.9 or greater, 1 or greater, 1.1 or greater, 1.2 or greater, 1.2 or greater, 1.4 or greater, 1.5 or greater, 1.6 or greater, 1.7 or greater, 1.8 or greater, 1.9 or greater, or 2.0 or greater. In some embodiments, the linker or X has a log P of from −0.3 to 4, from −0.2 to 4, from −0.1 to 4, from 0.1 to 4, from 0.1 to 3, or from 0.1 to 2.

In some examples, the linker or X can be a single amino acid, dipeptide, tripeptide, tetrapeptide, pentapeptide, hexapeptide, heptapeptide, octapeptide, nonapeptide, decapeptide, cyclic peptide, a peptide analog, or a combination thereof. In specific examples, the linker or X can comprise one or more of the following amino acids:

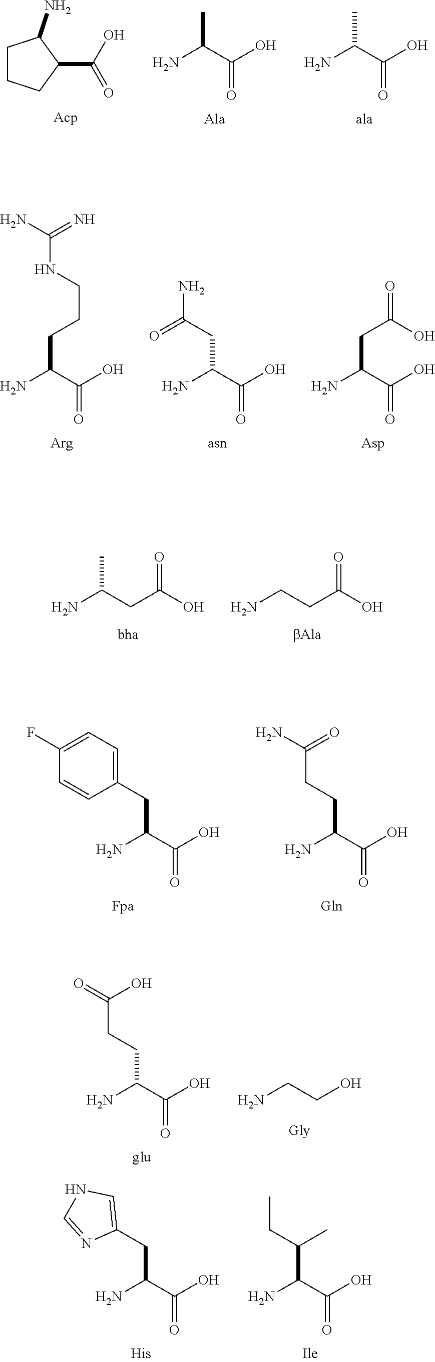

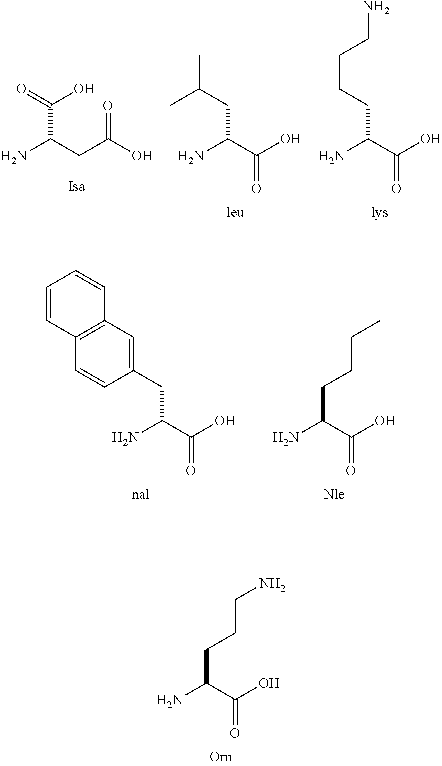

wherein the N- and C-terminus of the independently form peptide bonds. In preferred examples, the linker or X can comprise at least one non-polar amino acid selected from alanine, glycine, leucine, isoleucine, methionine, phenylalanine, proline, tryptophan, valine, or a combination thereof. In more preferred examples, the linker or X can comprise at least two non-polar amino acid selected from alanine, glycine, leucine, isoleucine, methionine, phenylalanine, proline, tryptophan, valine, or a combination thereof. In specific examples, the linker is not phenylalanine.

In further examples, the linker or X can comprise O, S, NH, R₄, OR₄, R₄O, OR₄O, C(O)R₄, R₄C(O), C(O)R₄C(O), C(O)OR₄, R₄OC(O), C(O)OR₄OC(O), C(O)R₄N, NR₄C(O), C(O)OR₄NH, NHR₄C(O)O, NHR₄, RANH, NHRANH, or C(O)NHR₄NHC(O), wherein R₄ is C₁-C₁₂ alkyl which can be optionally substituted with one or more substituents including halogen, alkoxyl, alkyl, alkenyl, cycloalkyl, cycloalkenyl, heterocycloalkyl, aryl, heteroaryl, amine, cyano, nitro, hydroxyl, carbonyl (C═O), acyl, carboxylic acid (—COOH), or amide (—CONH₂). In specific examples, the linker or X can be methyl, ethyl, propyl, butyl, pentyl, hexyl, heptyl, octyl, nonyl, or decyl. In other examples, the linker moiety or X can be polyethylene oxide (CH₂CH₂O)ₘ, where m is from 1 to 8.

In further examples, the disclosed compounds can have Formula II

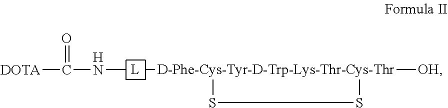

wherein L is Phe-Ala or Phe-Leu, or more specifically, D-Phe-D-Ala or D-Phe-D-Leu. In some examples, disclosed herein are compounds represented by the formula below:

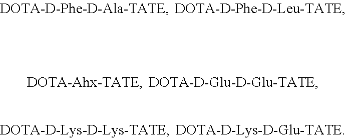

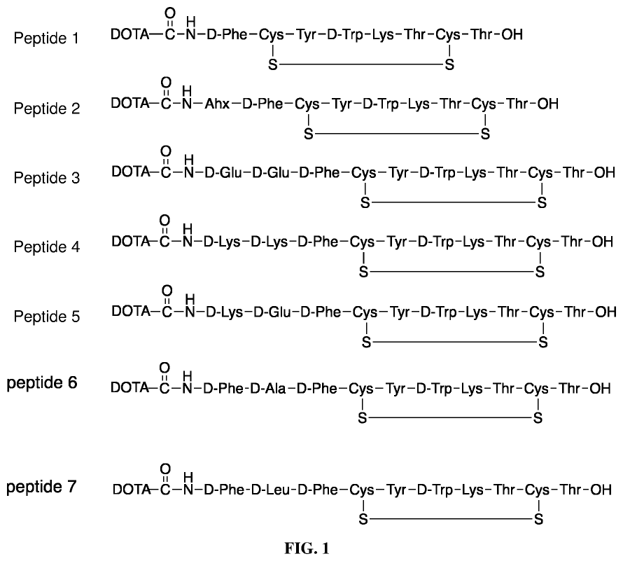

In specific examples, disclosed herein are compounds shown in FIG. 1 or Schemes 1-6.

The disclosed radiolabeled or non-radiolabeled compounds can be synthesized as exemplified in Schemes 1-6. Scheme 1 is exemplified immediately below.

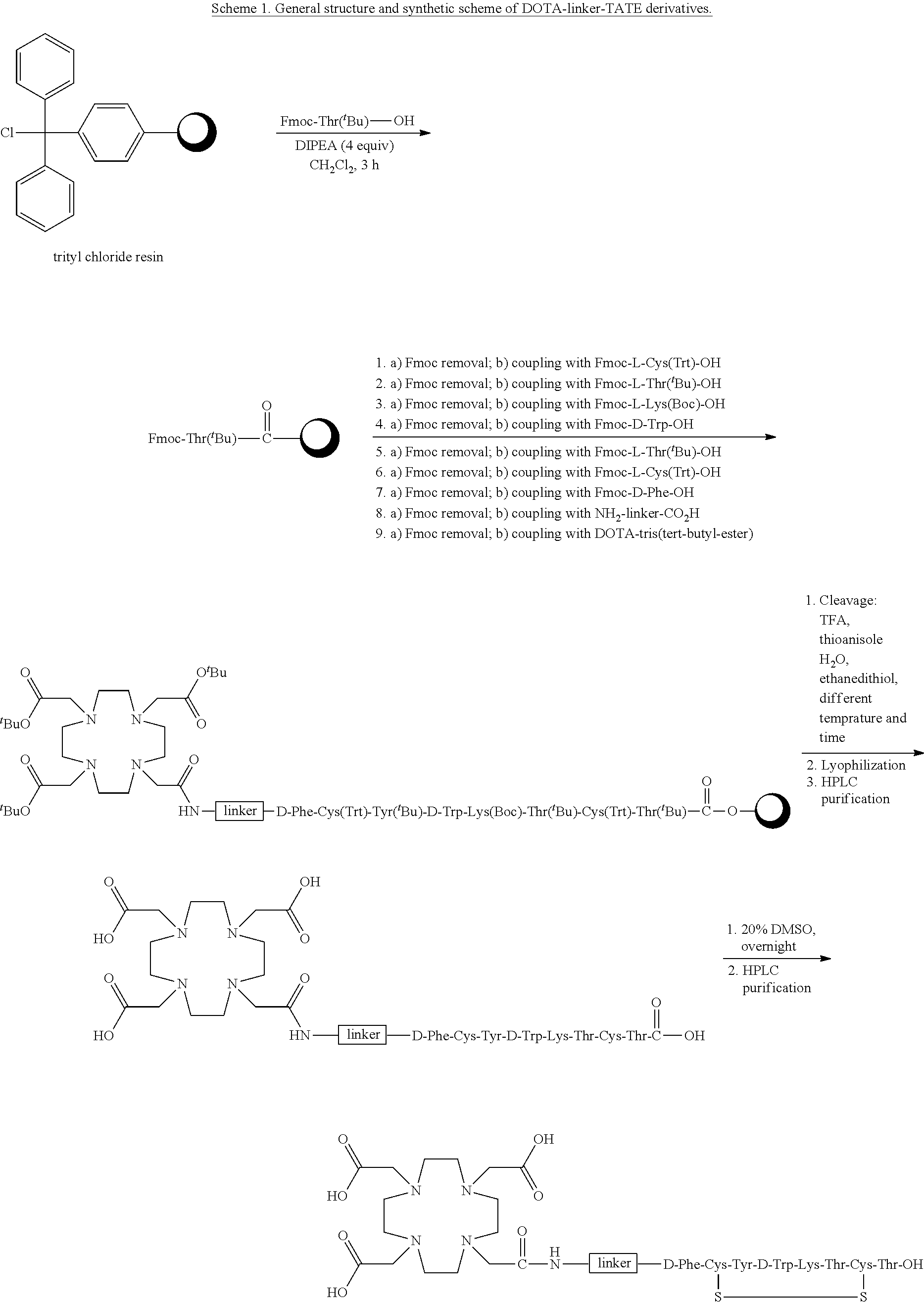

Pharmaceutical composition comprising a compound described herein, or pharmaceutically acceptable salt thereof, and a pharmaceutically acceptable carrier are also disclosed.

### Methods

Further provided herein are methods of treating or preventing a disorder of uncontrolled cellular proliferation, such as cancer in a subject, comprising administering to the subject an effective amount of a compound or composition as disclosed herein. The methods can further comprise administering a second compound or composition, such as, for example, anticancer agents or anti-inflammatory agents. Additionally, the method can further comprise administering an effective amount of ionizing radiation to the subject.

Methods of killing a tumor cell are also provided herein. The methods comprise contacting a tumor cell with an effective amount of a compound or composition as disclosed herein. The methods can further include administering a second compound or composition (e.g., an anticancer agent or an anti-inflammatory agent) or administering an effective amount of ionizing radiation to the subject.

The method can include administering the compound directly after removal of a primary tumor, upon detection of regions of tumor cells at the preangiogenic stage, upon diagnosis of high risk factors in the patient, or upon detection of certain cancer proteins in serum.

Also provided herein are methods of radiotherapy of tumors, comprising contacting the tumor with an effective amount of a compound or composition as disclosed herein and irradiating the tumor with an effective amount of ionizing radiation.

Also disclosed are methods for treating oncological disorders in a patient. In one embodiment, an effective amount of one or more compounds or compositions disclosed herein is administered to a patient having an oncological disorder and who is in need of treatment thereof. The disclosed methods can optionally include identifying a patient who is or can be in need of treatment of an oncological disorder. The patient can be a human or other mammal, such as a primate (monkey, chimpanzee, ape, etc.), dog, cat, cow, pig, or horse, or other animals having an oncological disorder. Oncological disorders include, but are not limited to, cancer and/or tumors of the anus, bile duct, bladder, bone, bone marrow, bowel (including colon and rectum), breast, eye, gall bladder, kidney, mouth, larynx, esophagus, stomach, testis, cervix, head, neck, ovary, lung, mesothelioma, neuroendocrine, penis, skin, spinal cord, thyroid, vagina, vulva, uterus, liver, muscle, pancreas, prostate, blood cells (including lymphocytes and other immune system cells), and brain. Specific cancers contemplated for treatment include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma. In specific examples, the oncological disorder are those cancers/tumors that express SSTR2. Such tumors are typically of neuroendocrine in origin.

Other examples of cancers that can be treated according to the methods disclosed herein are adrenocortical carcinoma, adrenocortical carcinoma, cerebellar astrocytoma, basal cell carcinoma, bile duct cancer, bladder cancer, bone cancer, brain tumor, breast cancer, Burkitt's lymphoma, carcinoid tumor, central nervous system lymphoma, cervical cancer, chronic myeloproliferative disorders, colon cancer, cutaneous T-cell lymphoma, endometrial cancer, ependymoma, esophageal cancer, gallbladder cancer, gastric (stomach) cancer, gastrointestinal carcinoid tumor, germ cell tumor, glioma, hairy cell leukemia, head and neck cancer, hepatocellular (liver) cancer, hypopharyngeal cancer, hypothalamic and visual pathway glioma, intraocular melanoma, retinoblastoma, islet cell carcinoma (endocrine pancreas), laryngeal cancer, lip and oral cavity cancer, liver cancer, medulloblastoma, Merkel cell carcinoma, squamous neck cancer with occult mycosis fungoides, myelodysplastic syndromes, myelogenous leukemia, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paranasal sinus and nasal cavity cancer, parathyroid cancer, penile cancer, pheochromocytoma, pineoblastoma and supratentorial primitive neuroectodermal tumor, pituitary tumor, plasma cell neoplasm/multiple myeloma, pleuropulmonary blastoma, prostate cancer, rectal cancer, renal cell (kidney) cancer, retinoblastoma, rhabdomyosarcoma, salivary gland cancer, Ewing's sarcoma, soft tissue sarcoma, Sezary syndrome, skin cancer, small cell lung cancer, small intestine cancer, supratentorial primitive neuroectodermal tumors, testicular cancer, thymic carcinoma, thymoma, thyroid cancer, transitional cell cancer of the renal pelvis and ureter, trophoblastic tumor, urethral cancer, uterine cancer, vaginal cancer, vulvar cancer, Waldenström's macroglobulinemia, and Wilms' tumor.

In some aspect, disclosed are methods for treating a breast cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanoma, glioma, leukemia, lymphoma, chronic myeloproliferative disorder, myelodysplastic syndrome, myeloproliferative neoplasm, and plasma cell neoplasm (myeloma), such as breast, prostate, ovarian and pancreatic cancer.

In some aspect, disclosed are methods for treating a tumor or tumor metastases in a subject by the administration to the subject a combination of at least one compound or composition as disclosed herein and at least one cancer immunotherapeutic agent. The disclosed compounds can be administered alone or in combination with a cancer immunotherapeutic agent. The subject can receive the therapeutic compositions prior to, during or after surgical intervention to remove all or part of a tumor. Administration may be accomplished via direct immersion; systemic or localized intravenous (i.v.), intraperitoneal (i.p.), subcutaneous (s.c.), intramuscular (i.m.), or direct injection into a tumor mass; and/or by oral administration of the appropriate formulations.

Methods for detecting tumor cells comprising administering to a patient a compound described herein and detecting the compound by imaging the patient are disclosed. The method can be for use in diagnosing prostate cancer and/or metastasis thereof.

### Administration

The disclosed compounds can be administered either sequentially or simultaneously in separate or combined pharmaceutical formulations. In some aspects, the compound is administered as an adjunctive therapeutic treatment. When one or more of the disclosed compounds is used in combination with a second therapeutic agent, the dose of each compound can be either the same as or differ from that when the compound is used alone. Appropriate doses will be readily appreciated by those skilled in the art.

The term “administration” and variants thereof (e.g., “administering” a compound) in reference to a compound as described herein means introducing the compound or a prodrug of the compound into the system of the animal in need of treatment. When a compound as described herein or prodrug thereof is provided in combination with one or more other active agents (e.g., a cytotoxic agent, etc.), “administration” and its variants are each understood to include concurrent and sequential introduction of the compound or prodrug thereof and other agents.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 2003/0032594 and 2002/0120100 disclose amino acid sequences that can be coupled to another composition and that allows the composition to be translocated across biological membranes. U.S. Application Publication No. 2002/0035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease. For example, the compounds can be used with mutant herpes simplex virus in the treatment of non-small cell lung cancer (Toyoizumi, et al., “Combined therapy with chemotherapeutic agents and herpes simplex virus type IICP34.5 mutant (HSV-1716) in human non-small cell lung cancer,” Human Gene Therapy, 1999, 10(18): 17).

Therapeutic application of compounds and/or compositions containing them can be accomplished by any suitable therapeutic method and technique presently or prospectively known to those skilled in the art. Further, compounds and compositions disclosed herein have use as starting materials or intermediates for the preparation of other useful compounds and compositions.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts, hydrates, or analogs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze-drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like. Drug delivery systems for delivery of pharmacological substances to dermal lesions can also be used, such as that described in U.S. Pat. No. 5,167,649.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver a compound to the skin are disclosed in U.S. Pat. Nos. 4,608,392; 4,992,478; 4,559,157; and 4,820,508.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art; for example, see U.S. Pat. No. 4,938,949.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

For the treatment of oncological disorders, compounds and agents and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds and agents and compositions disclosed herein can be used in methods of treating cancer wherein the patient is to be treated or is or has been treated with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation; East Hanover, NJ) and HERCEPTIN (Genentech, Inc.; South San Francisco, CA), respectively. These other substances or radiation treatments can be given at the same as or at different times from the compounds disclosed herein. Examples of other suitable chemotherapeutic agents include, but are not limited to, altretamine, bleomycin, bortezomib (VELCADE), busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib (IRESSA), gemcitabine, hydroxyurea, idarubicin, ifosfamide, imatinib (GLEEVEC), irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uracil, temozolomide, thiotepa, tioguanine/thioguanine, topotecan, treosulfan, vinblastine, vincristine, vindesine, vinorelbine. In an exemplified embodiment, the chemotherapeutic agent is melphalan. Examples of suitable immunotherapeutic agents include, but are not limited to, alemtuzumab, cetuximab (ERBITUX), gemtuzumab, iodine 131 tositumomab, rituximab, trastuzamab (HERCEPTIN). Cytotoxic agents include, for example, radioactive isotopes (e.g., I¹³¹, I¹²⁵, Y⁹⁰, p³², Bi²¹³, Ac²²⁵ etc.), and toxins of bacterial, fungal, plant, or animal origin (e.g., ricin, botulinum toxin, anthrax toxin, aflatoxin, jellyfish venoms (e.g., box jellyfish, etc.) Also disclosed are methods for treating an oncological disorder comprising administering an effective amount of a compound and/or agent disclosed herein prior to, subsequent to, and/or in combination with administration of a chemotherapeutic agent, an immunotherapeutic agent, a radiotherapeutic agent, or radiotherapy.

### EXAMPLES

### Example 1: Pre-clinical Evaluation of [²²⁵Ac]Ac-DOTA-TATE for Treatment of Lung Neuroendocrine Neoplasms

There is significant interest in the development of targeted alpha-particle therapies (TATs) for treatment of solid tumors. The metal chelator-peptide conjugate, DOTA-TATE, loaded with the β-particle emitting radionuclide ¹⁷⁷Lu ([¹⁷⁷Lu]Lu-DOTA-TATE) is now standard care for neuroendocrine tumors that express the somatostatin receptor 2 (SSTR2) target. A recent clinical study demonstrated efficacy of the corresponding [²²⁵Ac]Ac-DOTA-TATE in patients that were refractory to [¹⁷⁷Lu]Lu-DOTA-TATE. Tafreshi N K, et al. (Pre-clinical Evaluation of [225Ac]Ac-DOTA-TATE for Treatment of Lung Neuroendocrine Neoplasms. EJNMMI (DOI: 10.1007/s00259-021-05315-1)) reports the radiosynthesis, toxicity, biodistribution (BD), radiation dosimetry (RD), and efficacy of [²²⁵Ac]Ac-DOTA-TATE in small animal models of lung neuroendocrine neoplasms (NENs).

[²²⁵Ac]Ac-DOTA-TATE was synthesized and characterized for radiochemical yield, purity and stability. Non-tumor bearing BALB/c mice were tested for toxicity and BD. Efficacy was determined by single intravenous injection of [²²⁵Ac]Ac-DOTA-TATE into SCID mice bearing human SSTR2 positive H727 and H69 lung NENs. RD was calculated using the BD data.

[²²⁵Ac]Ac-DOTA-TATE was synthesized with 98% yield, 99.8% purity, and displayed 97% stability after 2 days incubation in human serum at 37° C. All animals in the toxicity study appeared healthy 5 months post injection with no indications of toxicity, except that animals that received ≥111 kBq of [²²⁵Ac]Ac-DOTA-TATE had chronic progressive nephropathy. BD studies revealed that the primary route of elimination is by the renal route. RD calculations determined pharmacokinetics parameters and absorbed α-emission dosages from ²²⁵Ac and its daughters. For both tumor models, a significant tumor growth delay and time-to-experimental endpoint were observed following a single administration of [²²⁵Ac]Ac-DOTA-TATE relative to controls.

These results suggest significant potential for the clinical translation of [225 Ac]Ac-DOTA-TATE for lung NENs.

### Example 2: Lipophilicity Determines Routes of Uptake and Clearance, and Toxicity of an Alpha-Particle Emitting Peptide Receptor Radiotherapy

Lipophilicity is explored in the biodistribution (BD), pharmacokinetics (PK), radiation dosimetry (RD) and toxicity of an internally administered targeted alpha-particle therapy (TAT) under development for the treatment of metastatic melanoma. The TAT is comprised of the DOTA (1,4,7,10-tetraazacyclododecane-1,4,7,10-tetraacetate) chelator conjugated to the melanocortin receptor 1 specific peptidic ligand (MCIRL) using a linker moiety and chelation of the ²²⁵Ac radiometal. A set of conjugates were prepared with a range of lipophilicities (LogD_{7.4} values) by varying the chemical properties of the linker. Reported are the observations that higher LogD_{7.4} values are associated with decreased kidney uptake, decreased absorbed radiation dose, and decreased kidney toxicity of the TAT, and the inverse is observed for lower LogD_{7.4} values. Animals administered TATs with lower lipophilicities exhibited acute nephropathy and death, whereas animals administered the highest activities of TATs with higher lipophilicities lived for the duration of the seven-month study and exhibited chronic progressive nephropathy. Changes in TAT lipophilicity were not associated with changes in liver uptake, dose, or toxicity. Significant observations include that lipophilicity correlates with kidney BD, the kidney-to-liver BD ratio, and weight loss; and that blood urea nitrogen (BUN) levels correlated with kidney uptake. Further, BUN was identified as having higher sensitivity and specificity of detection of kidney pathology and, the liver enzyme alkaline phosphatase (ALKP) had high sensitivity and specificity for detection of liver damage associated with the TAT. These findings suggest that tuning radiopharmaceutical lipophilicity can effectively modulate the level of kidney uptake to reduce morbidity and improve both safety and efficacy.

Tafreshi N K, et al. (ACS Pharm Transl Sci (DOI: 10.1021/acsptsci.1c00035)) details the effect of lipophilicity on routes of uptake and clearance, and toxicity of an alpha-particle emitting peptide receptor radiotherapy, which is hereby incorporated by reference. Briefly, lipophilicity is a major determinant of route of clearance of small molecule drugs due to the dependence of renal clearance on membrane permeability. However, this observation has not been fully substantiated for peptide conjugates. Mass can influence the route of clearance, i.e., molecules <50 kDa are removed from circulation via renal glomeruli, while larger macromolecules clear via the splenic and hepatic routes. With the goal of characterizing the relationship between lipophilicity and clearance route, we designed and synthesized a set of peptide-conjugate based radiopharmaceuticals for targeted alpha-particle therapy (TAT) with a range of lipophilicities (LogD_{7.4} values) and comparable mass values ranging from 1361 to 1618 MW without metal chelation. Also, unnatural amino acids are incorporated into the peptidic component of these compounds and high radiochemical stability has been previously demonstrated for the ²²⁵Ac-DOTA-Ahx-MCIRL radiopharmaceutical. Hence, peptide metabolism is likely a minor factor in determining the route of clearance. Herein, we report characterization of the BD, PK, RD and toxicity of these compounds in mice, and the relationships of LogD_{7.4} values with the route and PK of uptake and clearance of the administered TAT.

Since the lipophilicities of the compounds were adjusted by inserting linkers that have different structures and chemical properties between the peptide targeting moiety and the metal chelator, quality control characterizations were also performed. As a non-radioactive surrogate of ²²⁵Ac, La³⁺ chelates were used for these assays. The high binding affinity (low nM Ki) of the MCIRL peptide for the MCIR receptor and LogD_{7.4} values were confirmed for each compound.

For BD, PK, and RD determinations, each compound was labeled with ²²⁵Ac with high radiochemical yield and purity. When evaluating the BD, PK and RD data, general observations can be made that the metallated compounds with LogD_{7.4} values above −2.6 have lower kidney uptake rates, clear more by the hepatic route than renal, and have a lower radiation dose to the kidney when compared to the compounds with LogD_{7.4} values below −3.4, which clear more by the renal route, have higher kidney uptake rates and higher radiation dose to the kidney. This negative correlation of decreased kidney uptake and decreased radiation dose with increasing lipophilicity can be explained as a result of an increase in passive reabsorption in the kidney tubule, leading to less renal uptake.

Radiation-induced renal toxicity is a major concern in the therapeutic application of peptides labeled with radiometals. In this example, two distinct pathological features were observed; acute kidney damage and chronic kidney damage. Acute kidney failure was observed in animals that were administered the compounds with LogD_{7.4} values below −3.4 and was grossly characterized by pale, pitted, small and irregular shaped kidneys at necropsy, and by acute nephropathy via pathology examination. Acute kidney damage resulted in a rapid loss of kidney function and death within the week following treatment. Animals administered the compounds with LogD_{7.4} values above −2.6 lived for the duration of the 7-month study and exhibited chronic pathologies of the kidney and liver at high administered activities. The relative probabilities of pathology in the kidney and liver by administered activity were calculated for all cohorts. Weight loss and serum biomarkers of kidney and liver toxicity were also quantified, and weight loss was shown to have high sensitivity and specificity for detection of both kidney and liver pathology. While creatinine and BUN levels are typically used as biomarkers of kidney damage, BUN was identified in our study as having higher sensitivity and specificity of detection of kidney pathology. Of all liver enzymes tested, ALKP had both high sensitivity and high specificity for detection of liver damage. Statistical analyses identified significant relationships among LogD_{7.4} and kidney BD, the kidney-to-liver BD ratio, weight loss and kidney clearance rates. Weight loss also significantly correlated with kidney clearance rates. BUN levels were significantly correlated with kidney uptake and liver clearance rates.

The relationships among the LogD_{7.4} values of the La³⁺ chelated compounds and the kidney uptake, kidney total absorbed dose, and kidney toxicity values of the corresponding ²²⁵Ac chelated compounds were determined. It is notable that the compounds with LogD_{7.4} values above −2.6 have kidney uptake rates, total absorbed doses, and probabilities of toxicity that range from 3- to 100- fold lower than the compounds with LogD_{7.4} values below −3.4.

The arginine side chain in the MCIRL targeting ligand is likely to be positively charged in all of the peptide conjugates at pH 7.4. This likely gives an overall plus one charge to the metallated no-linker, Ahx linker, and D-Lys-D-Glu analogs and no overall charge for those same unmetallated analogs. The plus three metals, La and ²²⁵Ac, are balanced by the three carboxylates giving no overall charge for the DOTA-metal-chelated portion of the conjugates. The free DOTA-MCIRL species have three negative charges from the DOTA carboxylates and two positive charges from the protonation of two of the four tertiary amines in the 1,4,7,10-tetrazadodecane ring system. The metallated and unmetallated D-Lys-D-Lys MCIRL analogs are expected to have overall plus three or plus four charge, respectively, but they are less polar than the relevant D-Lys-D-Glu MCIRL analogs, which suggests significantly reduced basicity for the Lys side chains at physiological pH due to the overall charge density of D-Lys-D-Lys analogs.

All of the linkers used for conjugation are expected to be stable to metabolic changes and all of the conjugates have epsilon lysine-amide linkages which are unlikely to be substrates of proteolytic enzymes. D-amino acids were used in the polar linkers to inhibit possible proteolytic cleavage between the first and second amino acids in the linkers themselves.

In conclusion, by using different linker chemistry to increase the lipophilicity of a TAT radiopharmaceutical, we demonstrated that the kidney uptake rate can be significantly decreased, which also decreases the total absorbed dose and probability of toxicity to the kidney. Liver uptake, dose and toxicity were not significantly influenced by the increase in lipophilicity. Hence, there is significant potential to decrease the kidney toxicity of radiopharmaceuticals via medicinal chemistry approaches that adjust lipophilicity.

### Example 3: Targeted Alpha Particle Therapy for Somatostatin Receptor 2 Positive Neuroendocrine Tumors and Metastases

Ac-225 radiosynthesis have been optimized for an initial library of DOTATATE and derivatives and BD studies in non-tumor bearing BALB/c mice with and without co-injection of amino-acid have been performed. La³⁺ (the most chemically similar, non-radioactive element) have been chelated into each of the derivatives and their binding affinity and Log D7.4 determined. The Log D values in this example were lower than the ˜-2.6 level, for which primary liver clearance was previously observed for the melanocortin targeted agent, and all derivatives in the initial library were observed to clear primarily via the kidney. A follow-on library was constructed in the subsequent examples that have preferred log D values (in the range of −2.6 or higher). In vitro binding affinity (Kd) and radiochemical stability of 225 Ac-DOTATATE was also determined. Table 1 and FIG. 1 provides the structures and properties of the initial library of DOTATATE derivatives.

TABLE 1
Properties of DOTA-linker-TATE peptides with different linkers and chelates.

| | | | | | Radiolabeling | | |
|---|---|---|---|---|---|---|---|
| | | | | Kᵢ (nM) | yield, purity | Log D_{7.4} | Kidney/Liver |
| | | | HPLC | of La³⁺ | (%) of ²²⁵Ac | of La³⁺ | uptake ratio |
| # | Compound | MW | Purity | chelate | chelate | chelate | (24 h) |
| 1, 7 | DOTA-TATE | 1436 | 98% | 19.0 ± 9.2 | ≥98, ≥99.7 | −2.95 | 3.6 |
| 2, 8 | DOTA-Ahx-TATE | 1549 | 96% | 5.2 ± 0.4 | ≥98, ≥99.6 | −2.95 | 5.3 |
| 3, 9 | DOTA-di-D-Glu-TATE | 1694 | 97% | 37.2 ± 26.8 | ≥98, ≥99.5 | −3.16 | 16.5 |
| 4, 10 | DOTA-di-D-Lys-TATE | 1692 | 98% | 3.8 ± 2.8 | ≥98, ≥99.6 | −3.28 | 89.6 |
| 5, 11 | DOTA-D-Lys-D-Glu-TATE | 1693 | 97% | 46.9 ± 34.5 | ≥98, ≥99.7 | −3.10 | 87.6 |
| 12, 14 | DOTA-D-Phe-D-Ala-TATE | 1654 | 99.8% | 0.79 ± 0.60 | ≥98.7, ≥99.6 | −2.72 | ND |
| 13, 15 | DOTA-D-Phe-D-Leu-TATE | 1696 | 100% | 0.50 ± 0.35 | ≥98.8, ≥99.7 | −2.15 | ND |

Ahx: 6-amino hexanoic acid; D-Glu: D-glutamic acid; D-Lys: D-lysine; ND=not determined

Provided below, are methods for:

Chelation of La³⁺ and Eu³⁺ into the derivatives: To a solution of DOTA-linker-TATE derivatives in 0.1 M AcONa buffer (pH 6.0) was added LaCl₃ 7H₂O or EuC₁₃ 6H₂O. The mixture was stirred at room temperature for 12 h. After completion, the reaction was checked by HPLC, and the mixture went through a C18 column to yield the final product.

Log D7.4 measurements of the La³⁺-labeled derivatives: The octanol-water partition coefficients at pH7.4 (LogD_{7.4}) of DOTA-linker-TATE derivatives were determined using the miniaturized shake flask assay (Table 1). Briefly, 1:1 n-octanol (HPLC grade) was added to the solution of the test compound in PBS buffer (NaH₂PO₄/Na₂HPO₄ buffer, pH 7.4). The mixture was well mixed and then two layers were separated by centrifuge. The concentrations of compound in each layer were determined by HPLC.

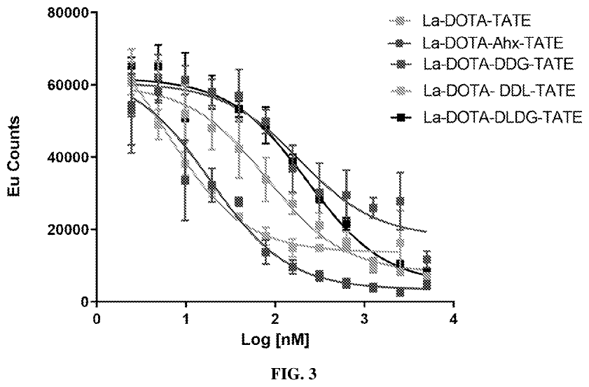

Binding assays of the La³⁺-labeled derivatives: As done previously, the Eu³⁺ chelate was used as a competing ligand for the La³⁺ chelate test agents using whole-cell competition binding assay (FIG. 3). Binding affinities are reported as Ki values.

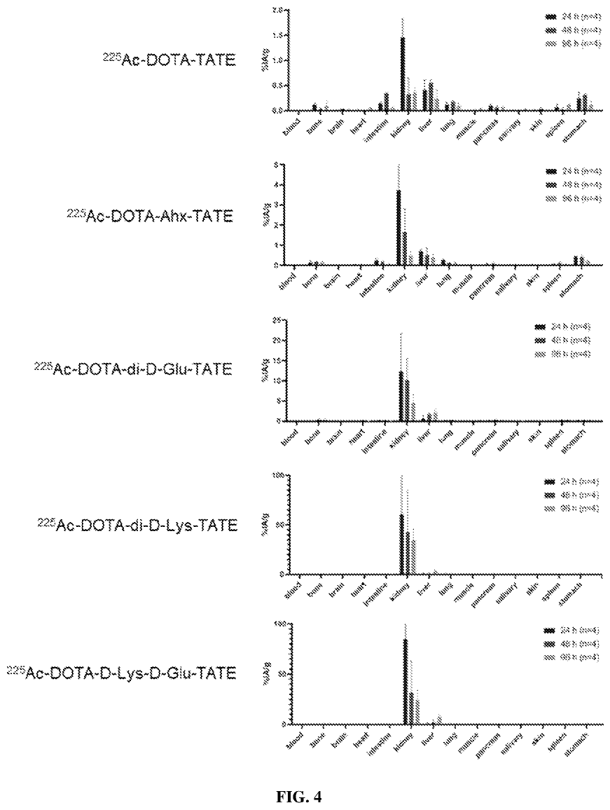

BD of ²²⁵Ac-DOTA-linker-TATE derivatives in non-tumor bearing BALB/c mice: The biodistribution studies for all 5 compounds were conducted (FIG. 4 and Table 1). All 5 compounds had higher kidney uptake relative to liver, i.e., kidney/liver uptake ratios >1. Note the general decrease in kidney uptake and relative increase in liver uptake in the higher Log D value derivatives compared to lower Log D value derivatives as indicated by the kidney/liver uptake ratios. The hypothesis was that if Log D values in the range of −2.6 could be achieved, then greater liver uptake relative to kidney would be observed. However, the Log D values were all≤-2.95.

In vitro radiochemical stability of ²²⁵Ac-DOTATATE: Radiochemical stability was determined by incubation at 37° C. in human plasma over time and using radio-TLC as previously described (Table 2). The compound was >95% intact after 5 days incubation.

TABLE 2
In vitro serum stability of ²²⁵Ac-DOTATATE.

| | % Intact |
|---|---|
| Day | TLC scanner |
| 0 | 100 |
| 1 | 100.44 ± 0.88 |
| 3 | 99.27 ± 0.29 |
| 5 | 96.74 ± 2.06 |
| 7 | 93.19 ± 1.50 |
| 10 | 90.29 ± 4.00 |

Synthesis of the second DOTA-linker-TATE derivative library: A follow-on library was produced and contain two compounds as shown below.

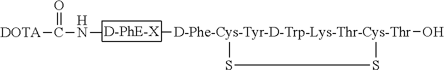

D-Phe was preserved to maintain the proper binding conformation; and X is a residue to adjust the physicochemical property of the resulting peptide. For the second library of two compounds, X=D-Ala and D-Leu.

### Example 4: Synthesis of Second DOTA-Linker-TATE Derivative Library

### Cycle 1 Synthesis 1

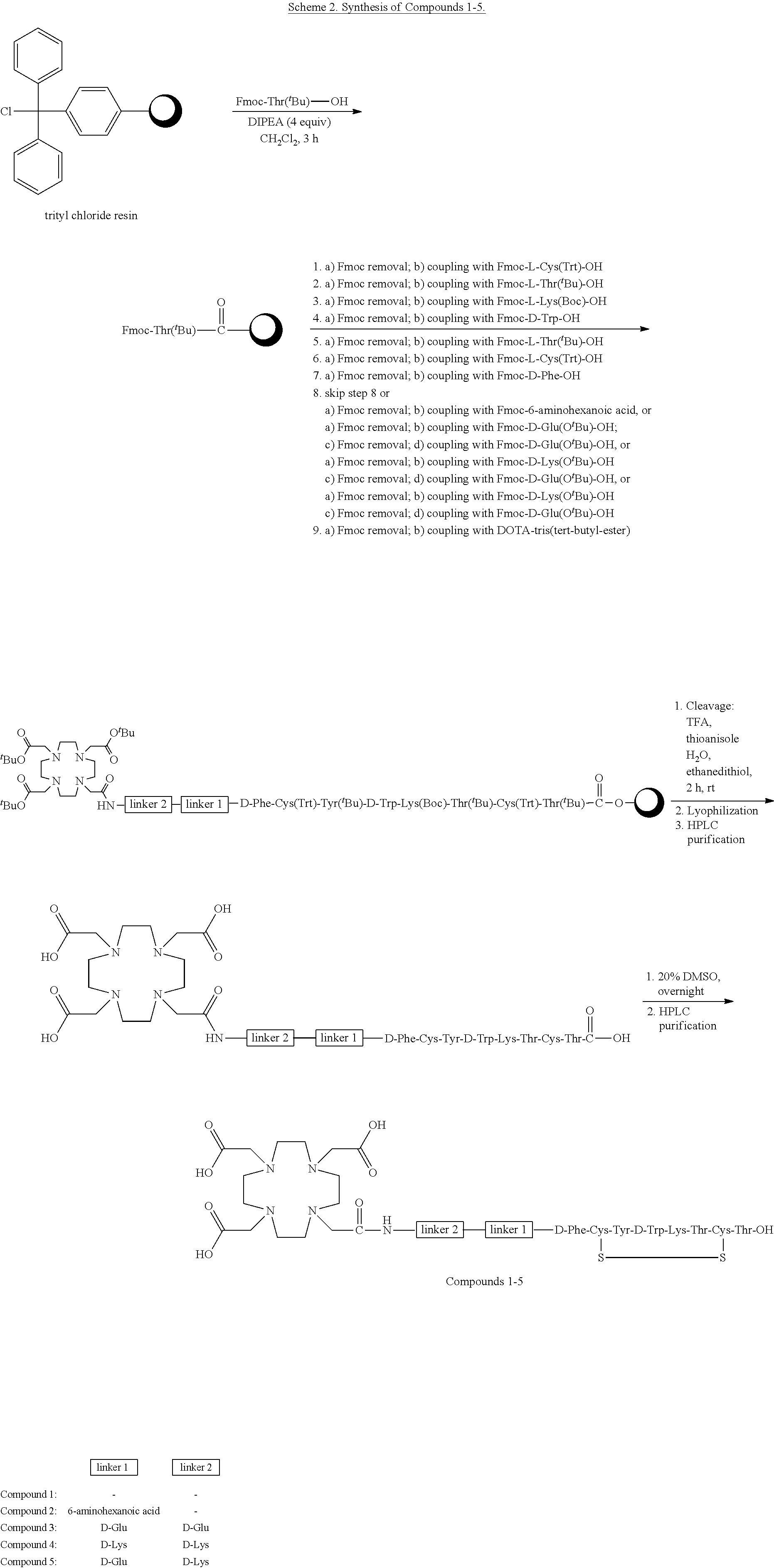

### Synthetic Procedure of Compounds 1-5.

Loading the trityl chloride resin. 1.0-1.6 mmol/g trityl chloride resin was allowed to react with 7.5×(7.5 equiv.) Fmoc-Thr(tBu)-OH in 25 mL of dry CH₂Cl₂ and 30 equiv. DIPEA for at least 8 h to load the trityle chloride resin. The unreacted resin was capped with methanol using CH₂Cl₂:MeOH:DIPEA (17:2:1).

Fmoc deprotection. Fmoc was deprotected from the amino acid on the resin by adding 5 mL of 20% piperidine. Nitrogen gas was bubbled in for 5 min to mix and the resulting mixture was washed 3 times with DMF from the solvent system.

Fmoc chemistry for peptide coupling. 7.5×(7.5 equiv.) Fmoc-amino acid (AA)-OH and O-(1H-6-chlorobenzotriazole-1-yl)-1,1,3,3-tetramethyluronium hexafluorophosphate (HCTU), 15×(15 equiv.) N,N-diisopropylethylamine (DIPEA). The coupling reaction was repeated twice for each position (25 min and 20 min, respectively). The amino acid building blocks used in the reaction are Fmoc-amino acid (AA)-OH: Fmoc-Cys(Trt)-OH for L-cysteine, Fmoc-Thr(tBu)-OH for L-threonine, Fmoc-Lys(Boc)-OH for L-lysine, Fmoc-D-Trp(Boc)-OH for D-tryptophan, Fmoc-Tyr(tBu)-OH, Fmoc-Phe-OH, 6-(Fmoc-amino) hexanoic acid (Ahx), and Fmoc-Glu(OtBu)-OH for glutamic acid (E).

Cleavage. After drying, the peptidyl resin was cleaved by the resin cleavage cocktail (90% TFA, 5% thioanisole, 2.5% H₂O, and 1.5% ethanedithiol) for 2 h at room temperature.

Purification. The target peptides were purified by the reversed phase HPLC twice using the Phenomenex Luna 5 uM C18(2) 100 Å preparative column. Mobile phase A: H₂O with 0.1% trifluoromethyl acetic acid (TFA). Mobile phase B: 80% acetonitrile with 0.1% TFA.

Formation of disulfide bond with DMSO. 20% aq. DMSO was added into the round bottom flask that contains the peptide and the reaction mixture was stirred for 24 h. The reaction was monitored by analytical HPLC and LC-MS. After the reaction was completed, the reaction mixture was concentrated and subjected to HPLC purification to obtain the final products.

### Results:

Compound 1 was obtained as a white powder. Chemical Formula: C₆₅H₉₀N₁₄O₁₉S₂. HPLC purity: 98%. HRMS (ESI): Calcd. for C₆₅H₉₀N₁₄O₁₉S₂: [M+H]⁺ 1435.6021, [M+Na]⁺1457.5840; found: 1435.6034 [M+H]⁺, 1457.5856 [M+Na]⁺.

Compound 2 was obtained as a white powder: Chemical Formula: C₇₁H₁₀₁N₁₅O₂₀S₂. HPLC purity: 96%. HRMS (ESI): Calcd. for C₇₁H₁₀₁N₁₅O₂₀S₂: [M+H]⁺ 1548.6785, [M+Na]⁺1570.6681; found: 1548.6813 [M+H]⁺, 1570.6551 [M+Na]⁺.

Compound 3 was obtained as a white powder: Chemical Formula: C₇₅H₁₀₄N₁₆O₂₅S₂. HPLC purity: 97%. HRMS (ESI): Calcd. for C₇₅H₁₀₄N₁₆O₂₅S₂: [M+H]⁺ 1693.6873, [M+Na]⁺1715.6692; found: 1693.6894 [M+H]⁺, 1715.6699 [M+Na]⁺.

Compound 4 was obtained as a white powder. Chemical Formula: C₇₇H₁₁₄N₁₈O₂₁S₂. HPLC purity: 98%. HRMS (ESI): Calcd. for C₇₇H₁₁₄N₁₈O₂₁S₂: [M+H]⁺ 1691.7920, [M+Na]⁺1713.7745; found: 1691.7941 [M+H]⁺, 1713.7771 [M+Na]⁺.

Compound 5 was obtained as a white powder. Chemical Formula: C₇₆H₁₀₉N₁₇O₂₃S₂. HPLC purity: 97%. HRMS (ESI): Calcd. for C₇₆H₁₀₉N₁₇O₂₃S₂: [M+H]⁺ 1692.7309, [M+Na]⁺1714.7221; found: 1692.7406 [M+H]⁺, 1714.7213 [M+Na]⁺.

### Cycle 1 Synthesis 2

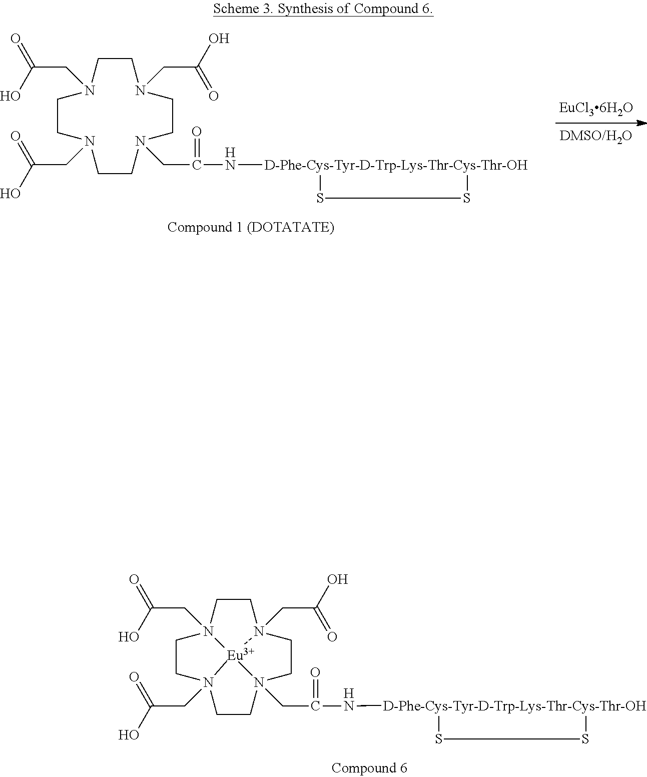

Synthetic Procedure: To a solution of compound 1 (DOTATATE) (2 mg) in 5 mL H₂O and 0.1 mL DMSO was added EuCl₃ 6H₂O (1.6 mg). The mixture was stirred at rt for 12 h. After completion of the reaction, the mixture was purified by the preparative HPLC column chromatography to yield the final product.

### Results:

Compound 6 was obtained as a white powder: Chemical Formula:

C₆₅H₃₆EuN₁₄O₁₉S₂. HPLC purity: 100%. HRMS (ESI) Calcd for C₆₅H₈₇EuN₁₄O₁₉S₂ (M+H)⁺ 1585.5004, found 1585.5031.

### Cycle 1 Synthesis 3

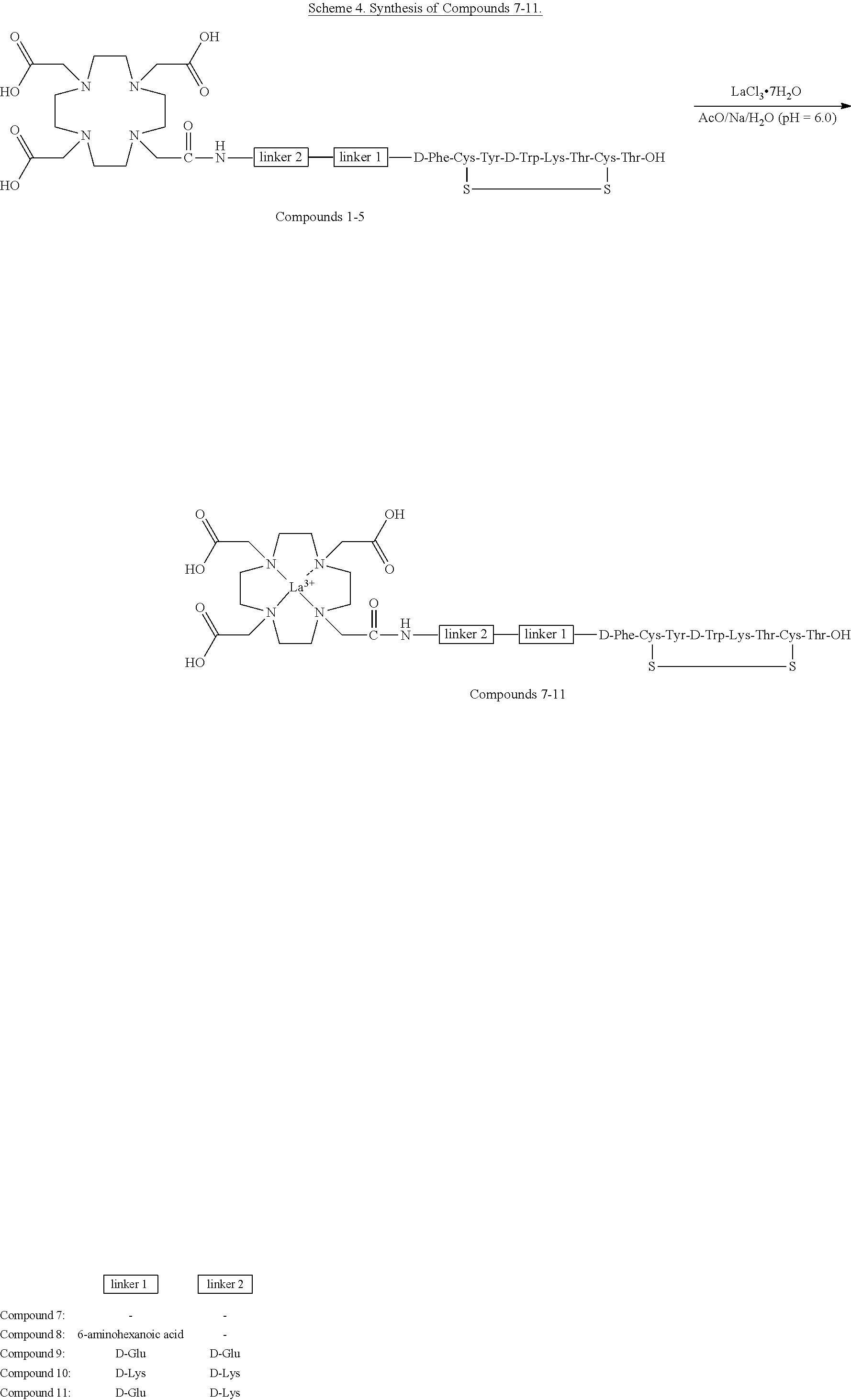

Synthetic Procedure: To a solution of compounds 1-5 (5 mg) in 1 mL 0.1 M AcONa buffer (pH 6.0) was added LaCl₃ 7H₂O (3.6 mg). The mixture was stirred at room temperature for 12 h. After completion of the reaction, the mixture was purified by the preparative HPLC column chromatography to yield the final products.

### Results

Compound 7 was obtained as a white powder. Chemical Formula:

C₆₅H₈₆EuN₁₄O₁₉S₂. HPLC purity: 100%. HRMS (ESI) Calcd for C₆₅H₈₇EuN₁₄O₁₉S₂ (M+H)⁺ 1571.4855, found 1571.4867.

Compound 8 was obtained as a white powder. Chemical Formula:

C₇₁H₉₇LaN₁₅O₂₀S₂. HPLC purity: 95.6%. HRMS (ESI) Calcd for C₇₁H₉₈LaN₁₅O₂₀S₂ (M+H)⁺ 1684.5696, found 1684.5676.

Compound 9 was obtained as a white powder. Chemical Formula:

C₇₅H₁₀₀LaN₁₆O₂₅S₂. HPLC purity: 97.0%. HRMS (ESI) Calcd for C₇₅H₁₀₁LaN₁₆O₂₅S₂ (M+H)⁺ 1829.5707, found 1829.5702.

Compound 10 was obtained as a white powder. Chemical Formula:

C₇₇H₁₁₀LaN₁₈O₂₁S₂. HPLC purity: 95.2%. HRMS (ESI) Calcd for C₇₇H₁₁₁LaN₁₈O₂₁S₂ (M+H)⁺ 1827.6754, found 1827.6716.

Compound 11 was obtained as a white powder. Chemical Formula:

C₇₆H₁₀₅LaN₁₇O₂₃S₂. HPLC purity: 98.4%. HRMS (ESI) Calcd for C₇₆H₁₀₆LaN₁₇O₂₃S₂ (M+H)⁺ 1828.6231, found 1828.6147.

### LogD_{7.4} Analysis

Compound 6: Eu-DOTA-TATE: LogD_{7.4}=−2.80

Compound 7: La-DOTA-TATE: LogD_{7.4}=−2.95

Compound 8: La-DOTA-Ahx-TATE: LogD_{7.4}=−2.95

Compound 9: La-DOTA-D-Glu-D-Glu-TATE: LogD_{7.4}=−3.16

Compound 10: La-DOTA-D-Lys-D-Lys-TATE: LogD_{7.4}=−3.28

Compound 11: La-DOTA-D-Lys-D-Glu-TATE: LogD_{7.4}=−3.10

### Cycle 2 Synthesis 1

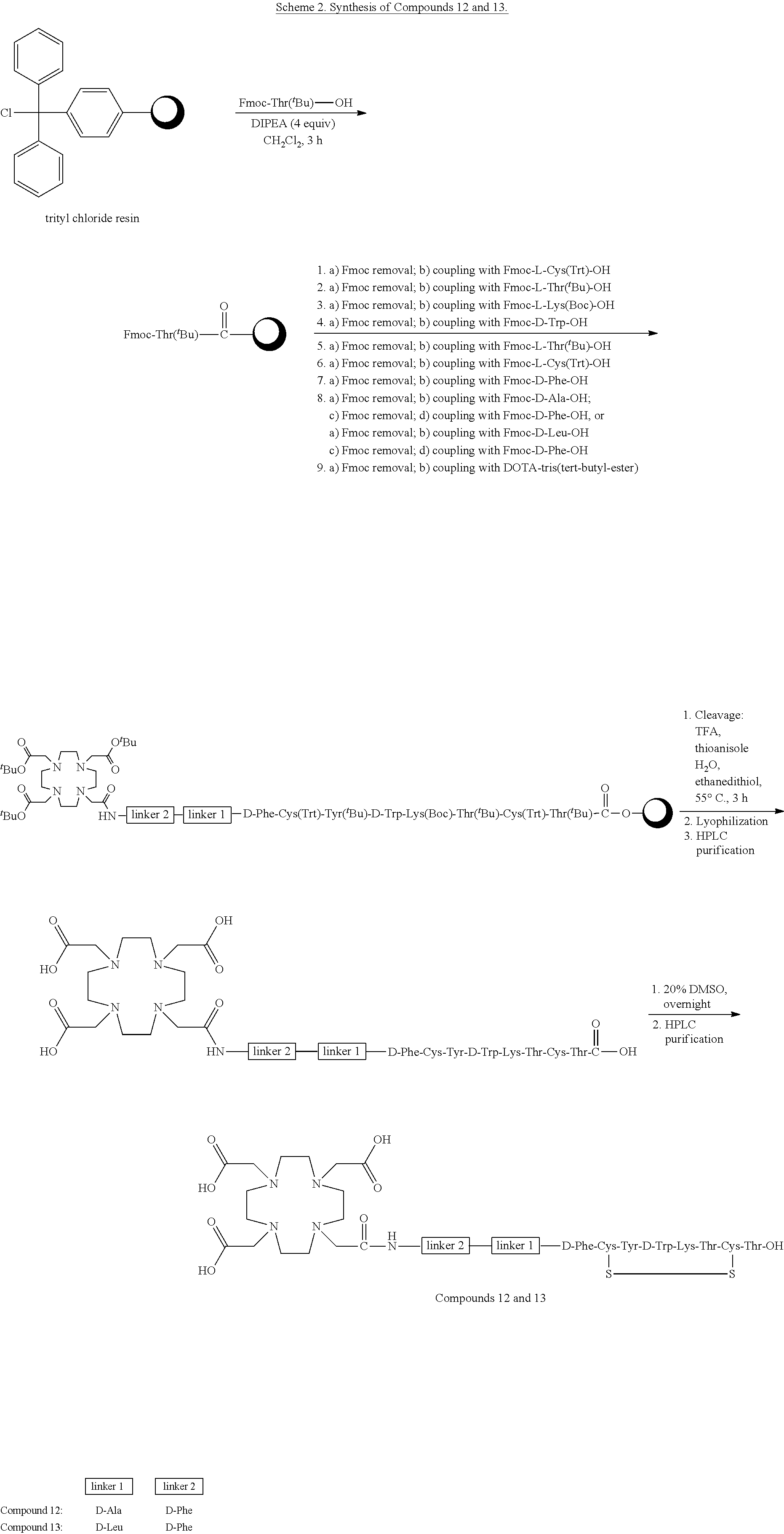

### Results:

Compound 12 was obtained as a white powder. Chemical Formula:

C₇₇H₁₀₃N₁₆O₂₁S₂. HPLC purity: 99.8%. HRMS (ESI) Calcd. for C₇₇H₁₀₄N₁₆O₂₁S₂: [M+H]⁺ 1653.7082, [M+Na]⁺1675.6901; found: 1653.7084 [M+H]⁺, 1675.6908 [M+Na]⁺.

Compound 13 was obtained as a white powder. Chemical Formula:

C₈₀H₁₀₉N₁₆O₂₁S₂. HPLC purity: 100%. HRMS (ESI) Calcd. for C₈₀H₁₁₀N₁₆O₂₁S₂: [M+H]⁺ 1695.7551, [M+Na]⁺1717.7371; found: 1695.7571 [M+H]⁺, 1717.7393 [M+Na]⁺.

### Cycle 2 Synthesis 2

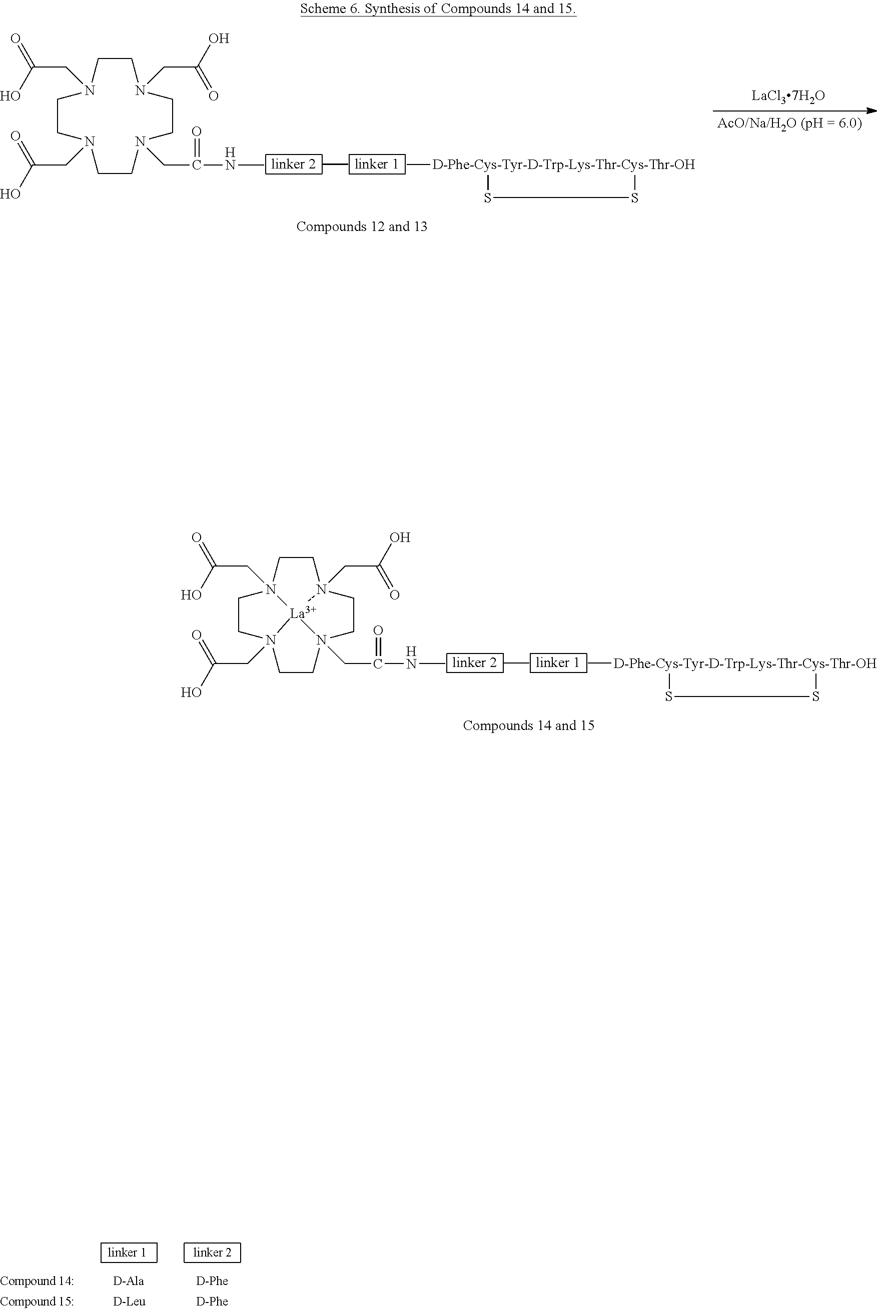

Compound 14 was obtained as a white powder. Chemical Formula:

C₇₇H₁₀₀LaN₁₆O₂₁S₂. HPLC purity: 100%. HRMS (ESI) Calcd for C₇₇H₁₀₁LaN₁₆O₂₁S₂ (M+H)⁺ 1789.5910, found 1789.5897.

Compound 15 was obtained as a white powder. Chemical Formula:

C₈₀H₁₀₆LaN₁₆O₂₁S₂. HPLC purity: 99.2%. HRMS (ESI) Calcd for C₈₀H₁₀₇LaN₁₆O₂₁S₂ (M+H)⁺ 1831.6380, found 1831.6352.

### LogD_{7.4} Analysis

Compound 14: 139La-DOTA-D-Phe-D-Ala-TATE: LogD_{7.4}: −2.72

Compound 15: 139La-DOTA-D-Phe-D-Leu-TATE: LogD_{7.4}: −2.15

### Determination of Binding Affinity of Compounds 14 and 15

Hek293 cells engineered to express SSTR2 were used to assess ligand binding in a competitive binding assay as described below. Note: Compound 14 (¹³⁹La-DOTA-D-Phe-D-Ala-TATE) is also referred to as ZW-7-123. Compound 15 (¹³⁹La-DOTA-D-Phe-D-Leu-TATE) is also referred to as ZW-7-124.

Black Corning 96-well plates (clear bottom, Corning, #3603) was coated with 0.25 mg/ml of PDL (Poly-D Lysin, Sigma), for 30 min in incubator, followed by washing with water (200 μl/well) two times. Hek293/SSTR2 cells were plated in the coated plates, at a density of 30,000 cells/well. Poly-D-lysine coated plates creates a uniform positive charge at the surface of the plastic, thereby facilitating cell attachment, growth, and differentiation.

On the day of the experiment (one day after seeding the cells), the medium was aspirated and 50 AL of non-labeled competing ligand was added to each well in a series of decreasing concentrations (ranging from ˜1 μM to 0.25 nM), followed by 50 μL of Eu-DOTATATE at 50 nM. Both labeled and non-labeled ligands were diluted in binding medium (DMEM, 1 mM 1,10-phenanthroline, 200 mg/L bacitracin, 0.5 mg/L leupeptin, 0.3% BSA). Cells were incubated with labeled and non-labeled ligands for 1 h at 37° C. in cell culture incubator. Following incubation, cells were washed two times with PBS buffer (200 μl/well). Because of the DOTA, the cells were then incubated with 50 μl/well, 2M HCl for 2 hours in 37C, then neutralized with 2 M NaOH (55 μl/well) and 115 μL of enhancement solution (PerkinElmer) was added to each well. Cells were incubated for an additional 30 min at 37° ° C. prior to reading. The plates were read on a PerkinElmer VICTORx4 2030 multilabel reader using the standard Eu time-resolved fluorescence (TRF) measurement (340 nm excitation, 400 us delay, and emission collection for 400 us at 615 nm).

Competition curves were analyzed with GraphPad Prism software using the sigmoidal dose-response (variable slope) classical equation for nonlinear regression analysis.

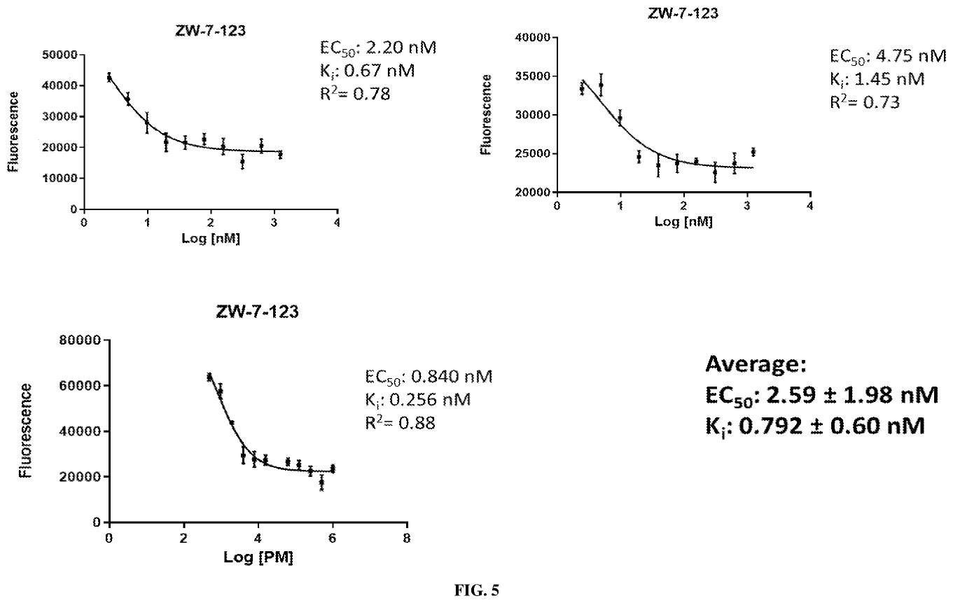

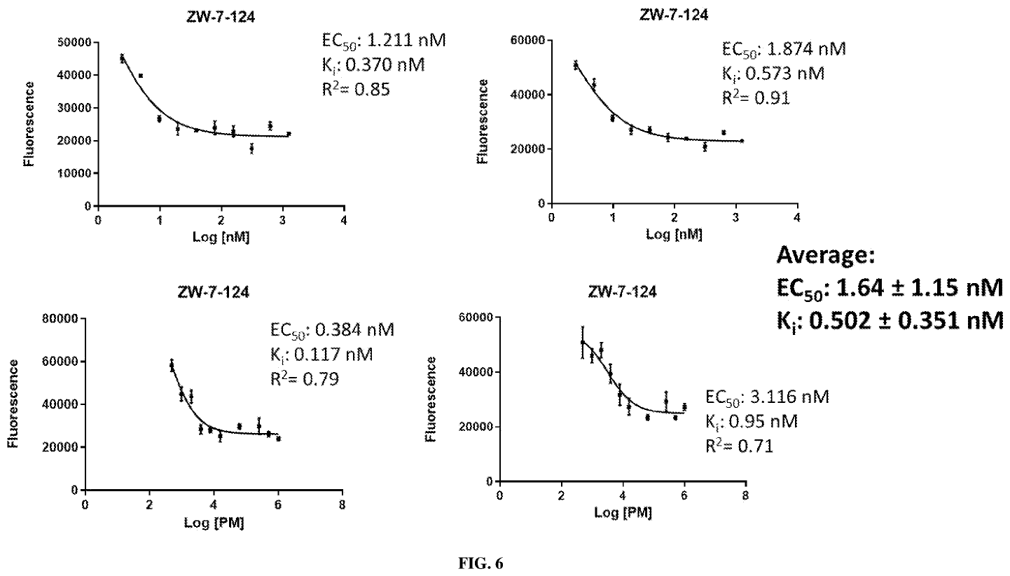

FIGS. 5 and 6 show the binding affinity of compounds 14 and 15 for the SSTR2 receptor and EC₅₀ values were confirmed for each compound, compounds 14 and 15 exhibited high binding affinity (low nM Ki) for the SSTR2 receptor.

1. A compound of Formula I: 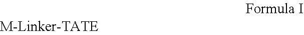
wherein
M is a macrocyclic radionuclide chelating moiety with the radionuclide optionally chelated therein;
Linker is a non-polar bridging moiety; and
TATE is 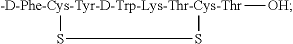
wherein the linker is not phenylalanine, and
wherein the compound has a Log D value of −2.7 or greater and/or wherein the linker has a Log D value of −0.3 or greater.

2. The compound of claim 1, wherein the Linker is from 1 to 100 atoms in length.

3. The compound of claim 1, wherein M is 2,2′,2″-(10-(2-((2,5-dioxopyrrolidin-1-yl)oxy)-2-oxoethyl)-1,4,7,10-tetraazacyclododecane-1,4,7-triyl)triacetic acid (DOTA).

4. The compound of claim 1, wherein the linker has a LogD of −0.2 or greater.

5. The compound of claim 1, wherein the linker comprises a single amino acid, dipeptide, tripeptide, tetrapeptide, pentapeptide, hexapeptide, heptapeptide, octapeptide, nonapeptide, decapeptide, cyclic peptide, a peptide analog, or a combination thereof.

6. The compound of claim 1, wherein the linker comprises at least one non-polar amino acid selected from alanine, glycine, leucine, isoleucine, methionine, phenylalanine, proline, tryptophan, valine, or a combination thereof.

7. The compound of claim 1, wherein the linker comprises at least two non-polar amino acid selected from alanine, glycine, leucine, isoleucine, methionine, phenylalanine, proline, tryptophan, valine, or a combination thereof.

8. The compound of claim 1, having a structure represented by Formula I-A: 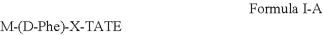
wherein
M is the macrocyclic radionuclide chelating moiety;
D-Phe is D-phenylalanine and maintains proper binding conformation of the compound to a receptor; and
X is from 1 to 100 atoms in length and comprises an organic moiety to adjust a physicochemical property of the compound.

9. The compound of claim 8, wherein the compound is represented by the formula 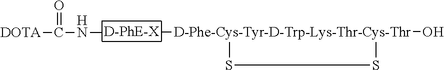
wherein D-Phe maintains proper binding conformation of the compound to a receptor; and
X is an organic moiety to adjust the physicochemical property of the compound.

10. The compound of claim 8, wherein X is selected from a natural or non-natural amino acid, a peptide or peptide mimetic, a polyethylene oxide, an optionally substituted amide, an optionally substituted ester, an optionally substituted C₁-C₁₂ alkyl, wherein the optional substituent can include one or more substituents selected from halogen, alkoxyl, alkyl, alkenyl, cycloalkyl, cycloalkenyl, heterocycloalkyl, aryl, heteroaryl, amine, cyano, nitro, hydroxyl, carbonyl (C═O), acyl, carboxylic acid (—COOH), or amide (—CONH₂).

11. The compound of claim 8, wherein X is selected from a natural or non-natural amino acid.

12. The compound of claim 8, wherein the compound is represented by the formula below:

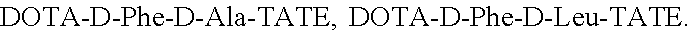

13. (canceled)

14. The compound of claim 1, wherein the macrocyclic radionuclide chelating moiety, M, includes the chelated radionuclide.

15. The compound of claim 14, wherein the radionuclide is selected from ⁸⁹Zr, ⁴⁴Sc, ¹¹¹In, ⁹⁰Y, ⁶⁸Ga, ¹⁷⁷Lu, ⁹⁹mTc, ⁶⁴Cu, ⁶⁷Cu, ¹⁵³Gd, ¹⁵⁵Gd, ¹⁵⁷Gd, ²¹³Bi, or ²²⁵Ac.

16. The compound of claim 14, wherein the radionuclide is ²²⁵Ac.

17. A complex comprising a radionuclide and a compound of claim 1.

18. A pharmaceutical composition comprising a compound of claim 1, or pharmaceutically acceptable salt thereof, and a pharmaceutically acceptable carrier.

19. A method of treating cancer in a subject comprising: administering to the subject a therapeutically effective amount of a compound of claim 1, or a pharmaceutically acceptable salt thereof.

20. (canceled)

21. The method of claim 19, wherein the cancer is selected from breast cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanoma, glioma, leukemia, lymphoma, chronic myeloproliferative disorder, myelodysplastic syndrome, myeloproliferative neoplasm, and plasma cell neoplasm (myeloma).

22. The method of claim 21, wherein the cancer is one of breast, prostate, ovarian and pancreatic cancer.

23. The method of claim 19, wherein the compound is administered as an adjunctive therapeutic treatment.

24. The method of claim 19, wherein the compound is administered directly after removal of a primary tumor, upon detection of regions of tumor cells at the preangiogenic stage, upon diagnosis of high risk factors in the patient, or upon detection of certain cancer proteins in serum.

25. (canceled)

26. (canceled)

27. A method for detecting tumor cells comprising: administering to a patient a compound of claim 1, and detecting the compound by imaging the patient.

28. The method of claim 27, wherein the compound is for use in a method of diagnosing prostate cancer and/or metastasis thereof.

29. (canceled)

30. (canceled)
